(19) United States
(12) Patent Application Publication
(10) Pub. No.: US 2026/0007396 A1
(43) Pub. Date: Jan. 8, 2026

(54) DEVICES AND METHODS FOR THE TREATMENT OF VASCULAR ABNORMALITIES

(71) Applicant: St. Jude Medical, Cardiology Division, Inc., St. Paul, MN (US)

(72) Inventors: Kristen Morin, St. Paul, MN (US); Trevor Springer, Maple Grove, MN (US); Andrey Kai, Plymouth, MN (US)

(73) Assignee: St. Jude Medical, Cardiology Division, Inc., St. Paul, MN (US)

(21) Appl. No.: 19/323,394

(22) Filed: Sep. 9, 2025

Related U.S. Application Data
(62) Division of application No. 18/455,002, filed on Aug. 24, 2023.
Application No. 18/455,002 is a continuation of application No. 17/391,666, filed on Aug. 2, 2021, now Pat. No. 11,771,410.
(60) Provisional application No. 63/060,221, filed on Aug. 3, 2020.

(51) Int. Cl. A61B 17/00; A61B 17/12

(52) U.S. Cl. CPC A61B 17/0057; A61B 17/12122; A61B 2017/00592; A61B 2017/00606; A61B 2017/00623; A61B 2017/00628; A61B 2017/00632; A61B 2017/00871

(57) ABSTRACT

Described herein is a medical device for treating a target site. The medical device includes a proximal end including a disc and a distal end including a lobe. The disc and lobe are connected by a connecting member. The disc includes a proximal surface, a distal surface, and a central surface extending between and connecting the proximal surface and distal surface, wherein the central surface separates the proximal surface from the distal surface by a predetermined depth distance.

### CROSS-REFERENCE TO RELATED APPLICATIONS

This application is a divisional of U.S. patent application Ser. No. 18/455,002, filed Aug. 24, 2023, which is a continuation of U.S. Pat. No. 11,771,410, filed Aug. 2, 2021, which claims the benefit of priority to U.S. Provisional Patent Application No. 63/060,221, filed Aug. 3, 2020, the entire contents of which are hereby incorporated by reference herein.

### BACKGROUND OF THE DISCLOSURE

### A. Field of Disclosure

The present disclosure relates generally to medical devices that are used in the human body. In particular, the present disclosure is directed to embodiments of an occlusion device that enables material apposition against tissue walls surrounding a vascular abnormality and facilitates a smooth transition between the tissue and the device. More specifically, the present disclosure is directed to an occlusion device with a disc having an increased depth, giving the disc a more three-dimensional cross-section. The embodiments and methods disclosed herein enable improved blood flow along or proximate to the disc after the occlusion device has been placed within the target site.

### B. Background

An occluder is a medical device used to treat (e.g., occlude) tissue at a target site within the human body, such as an abnormality, a vessel, an organ, an opening, a chamber, a channel, a hole, a cavity, a lumen, or the like. For example, an occluder may be used in Left Atrial Appendage (LAA) closures. LAAs are common heart defects in which there is a sac in the muscle wall of the left atrium. When a patient experiences atrial fibrillation (AFib), a blood clot may be formed in the LAA which may become dislodged and enter the blood stream. By occluding the LAA, the release of blood clots from the LAA may be significantly reduced, if not eliminated. Various techniques have been developed to occlude the LAA. For instance, balloon-like devices have been developed that are configured to be implanted completely within the cavity of the LAA, while surgical techniques have also been developed where the cavity of the LAA is inverted and surgically closed.

In the case of some known medical devices, a lobed portion of the device sits in a body of the LAA, and a disc portion is engaged with the opening of the LAA. The disc of these known devices is generally a two-dimensional shape, formed from two directly adjacent layers of metal fabric. With the diverse anatomy of LAAs, the implantation of a medical device designed to occlude the LAA may not result in optimal implantation, and the two-dimensional disc of the above described medical devices may potentially slip at least partially into the opening of the LAA, which may lead to areas of stagnant blood flow within the patient's vascular system. The presence of stagnant blood flow may increase the risk of a patient developing device related thrombosis (DRT), which may lead to further medical complications.

It would be advantageous to provide an improved occlusion device that reduces the risk of DRT by minimizing stagnant blood flow proximate to the deployed medical device.

### SUMMARY OF THE DISCLOSURE

The present disclosure generally relates to medical devices and methods of use thereof, including a disc with increased depth, giving the disc a more three-dimensional cross section which facilitates improved blood flow along or proximate to the disc after the occlusion device has been placed within the target site.

In one embodiment, the present disclosure is directed to a medical device for treating a target site. The medical device includes a proximal end and a distal end. The proximal end includes a disc and the distal end includes a lobe. The disc and the lobe are connected by a connecting member. The disc includes a proximal surface, a distal surface, and a central surface. The central surface extends between and connects the proximal surface and the distal surface. The central surface separates the proximal surface from the distal surface by a predetermined depth distance.

In another embodiment, a delivery system for deploying a medical device to a target site is provided. The delivery system includes a medical device and a delivery device. The medical device includes a proximal end and a distal end. The proximal end includes a disc and the distal end includes a lobe. The disc and the lobe are connected by a connecting member. The disc includes a proximal surface, a distal surface, and a central surface. The central surface extends between and connects the proximal surface and the distal surface. The central surface separates the proximal surface from the distal surface by a predetermined depth distance. The delivery device includes a delivery catheter, a delivery cable within the delivery catheter and translatable with respect to the delivery catheter, and a coupling member configured to couple the medical device to the delivery cable for facilitating at least one of deployment of the medical device at the target site and subsequent removal of the frame from the medical device.

In a further embodiment, a method for occluding a Left Atrial Appendage is provided. The method includes providing a medical device. The medical device includes a proximal end and a distal end. The proximal end includes a disc and the distal end includes a lobe. The disc and the lobe are connected by a connecting member. The disc includes a proximal surface, a distal surface, and a central surface. The central surface extends between and connects the proximal surface and the distal surface. The central surface separates the proximal surface from the distal surface by a predetermined depth distance. The method also includes advancing the medical device to the LAA using the delivery system, positioning the medical device relative to the LAA to occlude blood flow, and de-coupling the medical device from the delivery cable to deploy the medical device.

### BRIEF DESCRIPTION OF THE DRAWINGS

FIG. 1 is a schematic diagram of blood flow along or proximate to a known medical device, when the medical device is deployed at a target site.

FIG. 2 is a schematic diagram of a delivery system in accordance with the present disclosure;

FIG. 3 is a schematic diagram of blood flow along or proximate to medical device in accordance with the present disclosure, when the medical device is deployed at a target site;

FIGS. 4-8 illustrate various exemplary embodiments of the medical device, including three-dimensional discs having various shapes and configurations; and

FIG. 9 is a flow diagram of a method of occluding a target site within a patient's vascular system with a device in accordance with the present disclosure.

Corresponding reference characters indicate corresponding parts throughout the several views of the drawings. It is understood that that Figures are not necessarily to scale.

### DETAILED DESCRIPTION OF THE DISCLOSURE

The present disclosure relates generally to medical devices that are used in the human body. Specifically, the present disclosure provides medical devices, such as occlusion devices, including a distal lobe and a proximal disc, in which the disc has a defined depth, or a more three-dimensional shape than two-dimensional discs of at least some known medical devices.

Accordingly, the occlusion devices of the present disclosure facilitate improved and more uniform apposition of the disc against the tissue surrounding the vascular abnormality, which enables a smooth transition between the surrounding tissue and the medical device. Therefore, stagnant blood flow around the medical device or the abnormality may be reduced or eliminated.

The disclosed embodiments may lead to more consistent and improved patient outcomes. It is contemplated, however, that the described features and methods of the present disclosure as described herein may be incorporated into any number of systems as would be appreciated by one of ordinary skill in the art based on the disclosure herein.

Although the exemplary embodiment of the medical device is described as treating a target site including a left atrial appendage (LAA), it is understood that the use of the term “target site” is not meant to be limiting, as the medical device may be configured to treat any target site, such as an abnormality, a vessel, an organ, an opening, a chamber, a channel, a hole, a cavity, or the like, located anywhere in the body. The term “vascular abnormality,” as used herein is not meant to be limiting, as the medical device may be configured to bridge or otherwise support a variety of vascular abnormalities. For example, the vascular abnormality could be any abnormality that affects the shape of the native lumen, such as an atrial septal defect, an LAA, a lesion, a vessel dissection, or a tumor. Embodiments of the medical device may be useful, for example, for occluding an LAA, patent foreman ovalis (PFO), atrial septal defect (ASD), ventricular septal defect (VSD), or patent ductus arteriosus (PDA), as noted above. Furthermore, the term “lumen” is also not meant to be limiting, as the vascular abnormality may reside in a variety of locations within the vasculature, such as a vessel, an artery, a vein, a passageway, an organ, a cavity, or the like. As used herein, the term “proximal” refers to a part of the medical device or the delivery device that is closest to the operator, and the term “distal” refers to a part of the medical device or the delivery device that is farther from the operator at any given time as the medical device is being delivered through the delivery device. In addition, the terms “deployed” and “implanted” may be used interchangeably herein.

Some embodiments of the present disclosure provide an improved percutaneous catheter directed intravascular occlusion device for use in the vasculature in patients' bodies, such as blood vessels, channels, lumens, a hole through tissue, cavities, and the like, such as a left atrial appendage. Other physiologic conditions in the body occur where it is also desirous to occlude a vessel or other passageway to prevent blood flow into or therethrough. These device embodiments may be used anywhere in the vasculature where the anatomical conditions are appropriate for the design.

The medical device may include one or more layers of occlusive material, wherein each layer may be comprised of any material that is configured to substantially preclude or occlude the flow of blood so as to facilitate thrombosis. As used herein, “substantially preclude or occlude flow” shall mean, functionally, that blood flow may occur for a short time, but that the body's clotting mechanism or protein or other body deposits on the occlusive material results in occlusion or flow stoppage after this initial period.

The present disclosure now will be described more fully hereinafter with reference to the accompanying drawings, in which some, but not all embodiments of the disclosure are shown. Indeed, this disclosure may be embodied in many different forms and should not be construed as limited to the embodiments set forth herein; rather, these embodiments are provided so that this disclosure will satisfy applicable legal requirements. Like numbers refer to like elements throughout.

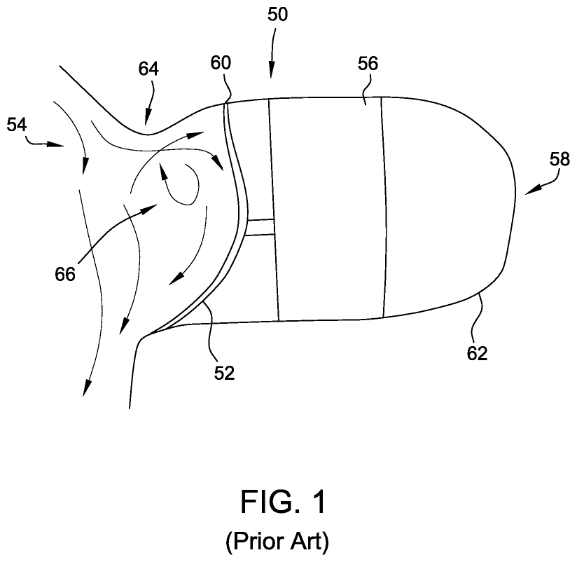

As shown in FIG. 1, at least some conventional or known medical devices 50 used for the occlusion of abnormalities include a disc 52 at a proximal end 54 and a lobe 56 at a distal end 58 thereof. Disc 52 is formed from an occlusive material that extends in a flat (e.g., two-dimensional) and planar configuration from a center of medical device 50 to a peripheral edge 60.

When known medical device 50 is deployed at a target site 62 (e.g., a left atrial appendage (LAA)), lobe 56 is positioned within the body of the LAA 62 and provides a layer of occlusion. Disc 52 is intended to abut the tissue at an opening 64 of the LAA and provides another layer of occlusion. Additionally, disc 52 prevents lobe 56 from shifting within the body of the LAA 62. When disc 52 has a two-dimensional configuration, disc 52 may recede at least partially into the body of the LAA 62, which may lead to areas 66 of stagnant flow in the region between opening 64 and the retracted edge 60 of disc 52, thereby increasing the risk of the patient developing DRT.

The medical devices of the present disclosure include a disc having a more three-dimensional shape, providing more depth and allowing for greater material apposition against the tissue at the opening to the abnormality while allowing for a smooth transition between the surrounding tissue and the medical device. The medical devices of the present disclosure thereby minimize the above-described disadvantages of known medical devices.

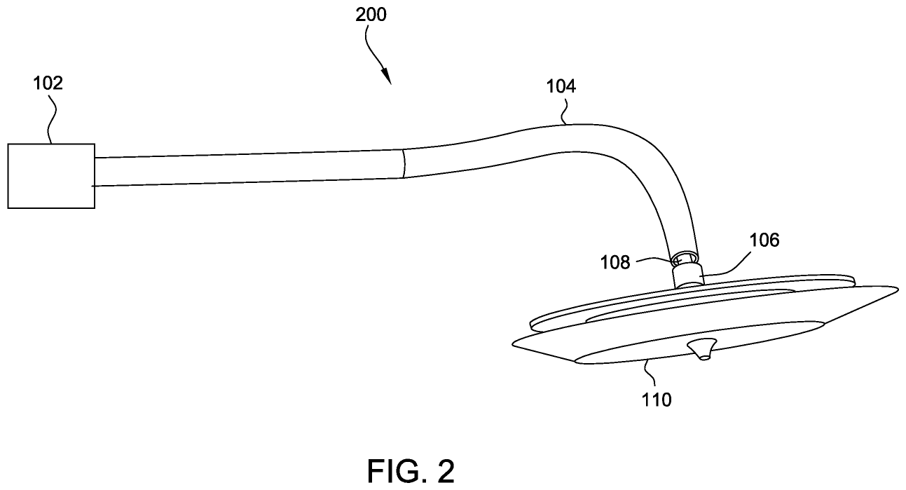

Turning now to FIG. 2, a schematic diagram of a delivery system 100 is shown. Delivery system 100 includes a delivery device 102 including a catheter 104 and a coupling member 106 configured to couple a distal end of a delivery cable 108 to a medical device 110 for facilitating the deployment of medical device 110 at a target site. Medical device 110 is deployed to treat the target site, and, in the example embodiment, is an occlusion device (“occluder”).

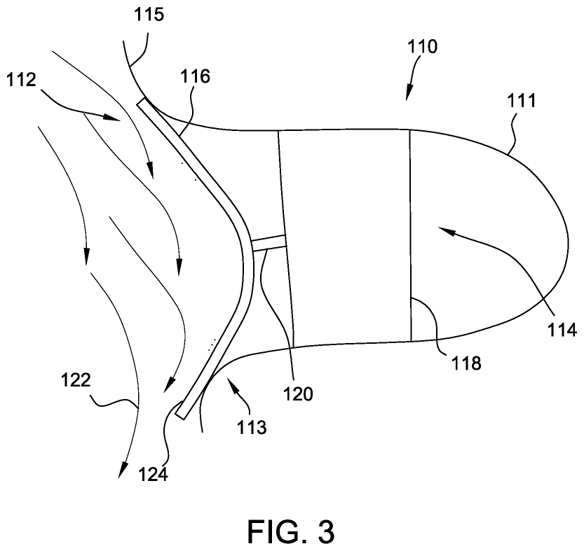

Turning now to FIG. 3, a schematic diagram of blood flow when medical device 110 according to the present disclosure is utilized for the occlusion of a target site 111 (e.g., an LAA) is shown. Medical device 110 includes a proximal end 112 and a distal end 114, wherein proximal end 112 includes a disc 116 and distal end 114 includes a lobe 118. Disc 116 is connected to lobe 118 by a connecting segment 120. When device 110 is deployed at the LAA 111, lobe 118 is positioned within the body of the LAA 111 and disc 116 is positioned at an opening 113 of the LAA 111. In the exemplary embodiment, disc 116 is formed with a three-dimensional shape, or having an appreciable depth, as described further herein, allowing for greater material apposition of disc 116 against tissue or walls 115 of the atrium around the opening 113 to the LAA 111, while maintaining a smooth transition between the walls 115 of the heart and disc 116. This increased material apposition decreases the risk of stagnant blood flow and ensures normal blood flow in a flow direction 122 proximate to disc 116 (e.g., along a proximal surface 124 thereof).

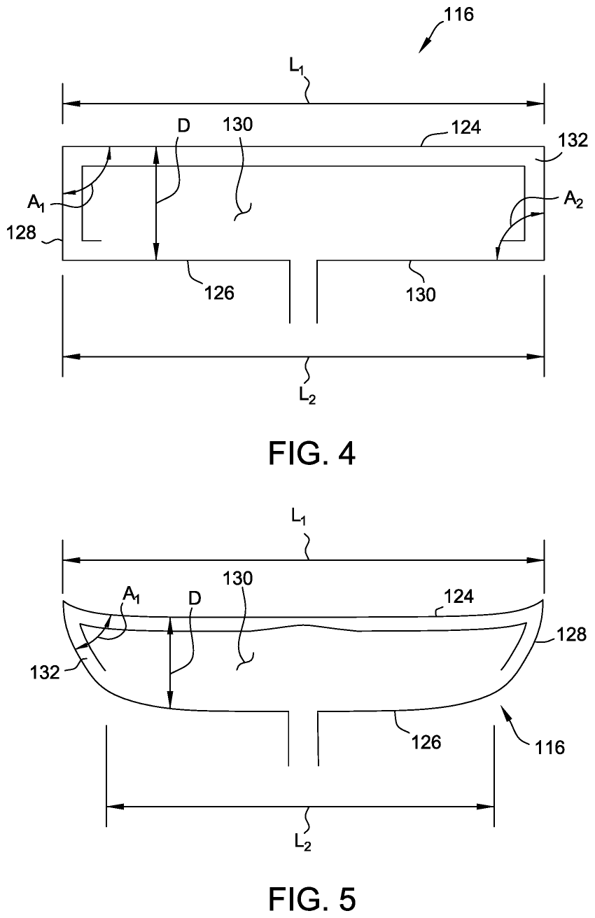

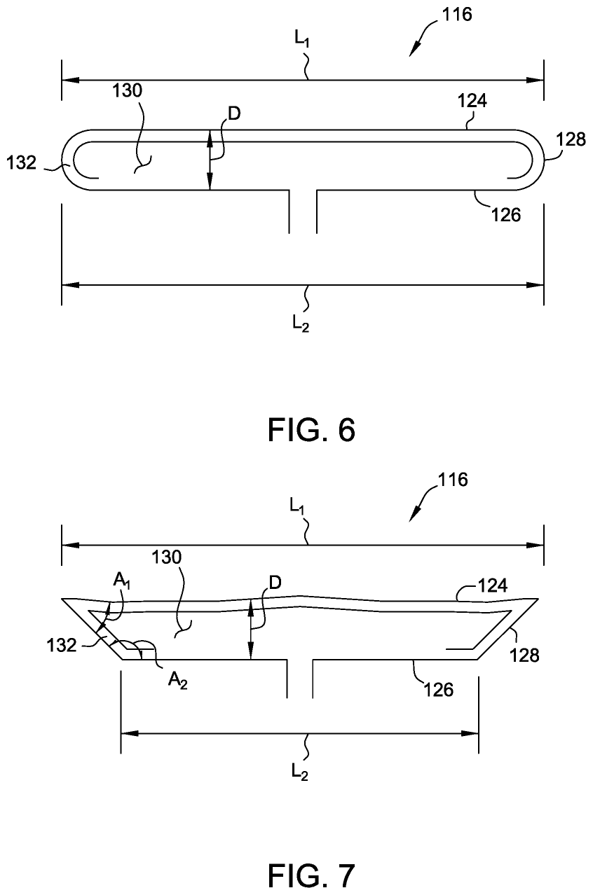

FIGS. 4-7 illustrate respective exemplary embodiments of disc 116 of medical device 110. In these embodiments, an entirety of disc 116 is three-dimensional in shape or has an appreciable depth. Specifically, FIG. 4 is a cross sectional view of disc 116 formed with a rectangular three-dimensional shape, FIG. 5 is a cross sectional view of disc 116 formed with a half-rounded (or partially rounded) three-dimensional shape, FIG. 6 is a cross sectional view of disc 116 formed with a fully rounded shape, and FIG. 7 is a cross sectional view of disc 116 formed with a trapezoidal three-dimensional shape.

As shown in FIGS. 4-7, disc 116 includes a proximal surface 124 having a first length (or diameter) L₁, a distal surface 126 having a second length (or diameter) L₂, and a central surface 128 (also referred to as an edge surface or edge). Central surface 128 extends between and connects proximal surface 124 and distal surface 126. A depth D of disc 116 is defined between proximal surface 124 and distal surface 126. In some embodiments, a length of central surface 128 corresponds to or defines depth D. In some embodiments, such as the embodiment of disc 116 shown in FIG. 4, depth D is defined consistently along the entirety of disc 116. In other embodiments, depth D varies along disc 116. Depth D may be 0.006 to 0.2 inches, more particularly 0.125 to 0.2 inches or 1 mm to 10 mm.

Proximal surface 124, distal surface 126, and central surface 128 define a cavity 130 of disc 116 therebetween. In the exemplary embodiment, disc 116 (and the rest of medical device 110) is formed from an occlusive fabric 132, and cavity 130 is defined between sheets of fabric 132 forming proximal surface 124 and distal surface 126 (which may be each be defined by a single layer or a double layer of fabric 132). That is, depth D represents a depth distance defined between these surfaces 124, 126 and by which surface 124, 126 are separated.

As shown in FIGS. 4 and 6, first length L₁ (or diameter) of proximal surface 124 may be approximately equal to second length L₂ (or diameter) of distal surface 126. As shown in FIGS. 5 and 7, first length L₁ of proximal surface 124 may be greater than second length L₂ of distal surface 126.

In some embodiments, central surface 128 extends linearly between proximal surface 124 and distal surface 126 (see, e.g., FIGS. 4 and 7). Central surface 128 may adjoin proximal surface 124 at an angle A₁. Angle A₁ may be approximately equal to a 90 degree angle or may be less than 90 degrees. Central surface 128 may adjoin distal surface 126 at an angle A₂. Angle A₂ may be greater than or equal to about 90 degrees.

Central surface 128 may alternatively adjoin proximal surface 124 and/or distal surface 126 with an angular or arcuate (e.g., curved) connection (see, e.g., FIGS. 5 and 6). It is also contemplated that central surface 128 may be curved between proximal surface 124 and distal surface 126 (see, e.g., FIGS. 5 and 6). Central surface 128 may have a radius of curvature. The radius of curvature may be in a range between 0.003 to 0.1 inches, more particularly 0.0625 to 0.1 inches or 2 mm to 10 mm. The radius of curvature may vary to ensure proper occlusive function and/or positioning of disc 116 within opening 113, while maintaining normal blood flow along or proximate to disc 116.

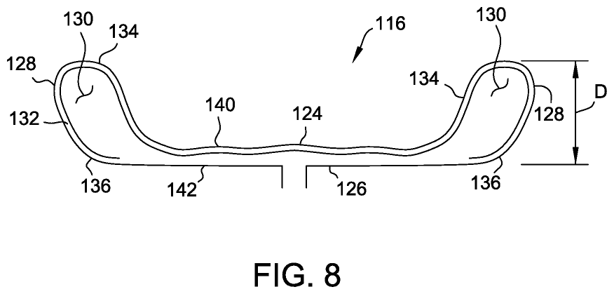

FIG. 8 illustrates another embodiment of disc 116 of medical device 110, in which portions of disc 116 are three-dimensional. Proximal surface 124 includes a proximal peripheral portion 134. Distal surface 126 includes a distal peripheral portion 136. Central surface 128 extends between and connects proximal peripheral portion 134 and distal peripheral portion 136. In some embodiments, proximal peripheral portion 134 and/or distal peripheral portion 136 extends in a proximal longitudinal direction.

In the embodiment of FIG. 8, cavity 130 is defined along the periphery of disc 116, as an annular cavity 130. That is, proximal surface 124 and distal surface 126 are spaced apart only along the periphery of disc 116. The depth distance D, defined between proximal surface 124 and distal surface 126 at peripheral portions 134, 136 thereof, provides a three-dimensional shape to the peripheral edges of disc 116. A central portion 140 of proximal surface 124, radially inward of peripheral portion 134, is directly adjacent to a central portion 142 of distal surface 126, radially inward of peripheral portion 136 (e.g., no cavity 130 is defined between surfaces 124, 126 in this central portion).

In one embodiment, medical device 110 is formed from a shape-memory material. One particular shape memory material that may be used is Nitinol. Nitinol alloys are highly elastic and are said to be “superelastic,” or “pseudoelastic.” This elasticity may allow medical device 110 to be resilient and return to a preset, expanded configuration for deployment following passage in a distorted form through delivery catheter 104. Further examples of materials and manufacturing methods for medical devices with shape memory properties are provided in U.S. Publication No. 2007/0265656 titled “Multi-layer Braided Structures for Occluding Vascular Defects” and filed on Jun. 21, 2007, which is incorporated by reference herein in its entirety.

It is also understood that medical device may be formed from various materials other than Nitinol that have elastic properties, such as stainless steel, trade named alloys such as Elgiloy®, or Hastalloy, Phynox®, MP35N, CoCrMo alloys, metal, polymers, or a mixture of metal(s) and polymer(s). Suitable polymers may include PET (Dacron™), polyester, polypropylene, polyethylene, HDPE, Pebax, nylon, polyurethane, silicone, PTFE, polyolefins and ePTFE. Additionally, it is contemplated that the medical device may comprise any material that has the desired elastic properties to ensure that the device may be deployed, function as an occluder, and be recaptured in a manner disclosed within this application.

In some embodiments, disc 116 is shaped as desired by heat-setting fabric 132 over a mandrel having a complementary shape. Additionally or alternatively, disc 116 may include a frame therein, where the frame has the desired final shape of disc 116. Fabric 132 may be coupled to this frame to form disc 116.

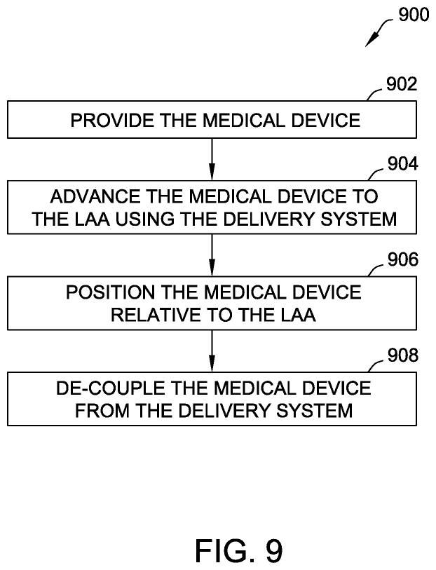

Turning now to FIG. 9, a flow diagram of an exemplary method 900 of using medical device 110 to occlude an LAA in a patient is shown. In the exemplary embodiment, method 900 includes providing 902 a medical device. As described herein, the medical device includes a proximal end and a distal end, wherein the proximal end comprises a disc and the distal end comprises a lobe, wherein the disc and lobe are connected by a connecting member, wherein the disc comprises a proximal surface, a distal surface, and a central surface extending between and connecting the proximal surface and distal surface, wherein the central surface separates the proximal surface from the distal surface by a predetermined depth distance.

Method 900 also includes advancing 904 the medical device to the LAA using a delivery system including a catheter and a delivery cable, positioning 906 the medical device relative to the LAA to occlude blood flow to and from the LAA, and de-coupling 908 the medical device from the delivery cable to deploy the medical device

Method 900 may include additional, alternative, and/or fewer steps, including those described herein. For example, in some embodiments, positioning 906 the medical device relative to the LAA includes placing the lobe of the medical device within the body of the LAA and the disc outside of the LAA to abut the adjacent wall surrounding the opening of the LAA.

Additionally, de-coupling 908 the medical device from the delivery cable includes transitioning the medical device from the constricted configuration adopted for delivery from a catheter to the preset expanded configuration.

While embodiments of the present disclosure have been described, it should be understood that various changes, adaptations and modifications may be made therein without departing from the spirit of the disclosure and the scope of the appended claims. Further, all directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. It is intended that all matter contained in the above description or shown in the accompanying drawings shall be interpreted as illustrative only and not limiting. Changes in detail or structure may be made without departing from the spirit of the disclosure as defined in the appended claims.

Many modifications and other embodiments of the disclosure set forth herein will come to mind to one skilled in the art to which this disclosure pertains having the benefit of the teachings presented in the foregoing descriptions and the associated drawings. Therefore, it is to be understood that the disclosure is not to be limited to the specific embodiments described and that modifications and other embodiments are intended to be included within the scope of the appended claims. Although specific terms are employed herein, they are used in a generic and descriptive sense only and not for purposes of limitation.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. An occluder for occluding a left atrial appendage (“LAA”), the occluder comprising:
a lobe positioned at a distal end of the occluder and configured to be positioned within a body of the LAA;
a disc positioned at a proximal end of the occluder and configured to abut tissue at an opening of the LAA; and
a connecting segment that connects the disc to the lobe and spaces apart the disc from the lobe;
wherein the occluder has a collapsed condition when received within a delivery catheter and a pre-set expanded condition in the absence of applied forces;
wherein the disc includes a proximal surface including a proximal peripheral portion, and a distal surface including a distal peripheral portion, the proximal and distal peripheral portions extending in a proximal longitudinal direction in the pre-set expanded condition, the distal peripheral portion of the distal surface separated from the proximal peripheral portion of the proximal surface by a depth such that an enclosed annular cavity is defined along a periphery of the disc between the proximal peripheral portion of the proximal surface and the distal peripheral portion of the distal surface, the depth being between 3.175 mm and 5.08 mm,
wherein the disc includes a side surface extending between the proximal peripheral portion of the proximal surface and the distal peripheral portion of the distal surface,
wherein the side surface includes a radius of curvature between 2 mm and 10 mm,
wherein, in an implanted condition of the occluder in which the lobe is positioned within the body of the LAA and the disc abuts tissue at the opening of the LAA, the depth of the disc and the radius of curvature of the side surface of the disc are configured to prevent the disc from receding into the body of the LAA while preventing blood flow from stagnating along the proximal surface of the disc.

2. The occluder of claim 1, wherein the occluder is formed of nitinol.

3. The occluder of claim 2, wherein the disc is formed as a heat-set fabric.

4. The occluder of claim 2, wherein the disc includes a frame.

5. The occluder of claim 4, wherein the disc includes a fabric coupled to the frame.

6. A system for occluding a left atrial appendage (“LAA”), the system comprising:
an occluder comprising:
a lobe positioned at a distal end of the occluder and configured to be positioned within a body of the LAA, a disc positioned at a proximal end of the occluder and configured to abut tissue at an opening of the LAA, and a connecting segment that connects the disc to the lobe and spaces apart the disc from the lobe; and
a delivery device comprising:
a delivery catheter;
a delivery cable within the delivery catheter and translatable with respect to the delivery catheter; and
a coupling member configured to couple the occluder to the delivery cable for facilitating at least one deployment of the occluder at the LAA,
wherein the occluder has a collapsed condition when received within the delivery catheter and a pre-set expanded condition in the absence of applied forces;
wherein the disc includes a proximal surface including a proximal peripheral portion, and a distal surface including a distal peripheral portion, the proximal and distal peripheral portions extending in a proximal longitudinal direction in the pre-set expanded condition, the distal peripheral portion of the distal surface separated from the proximal peripheral portion of the proximal surface by a depth such that an enclosed annular cavity is defined along a periphery of the disc between the proximal peripheral portion of the proximal surface and the distal peripheral portion of the distal surface, the depth being between 3.175 mm and 5.08 mm,
wherein the disc includes a side surface extending between proximal peripheral portion of the proximal surface and the distal peripheral portion of the distal surface,
wherein the side surface includes a radius of curvature between 2 mm and 10 mm,
wherein, in an implanted condition of the occluder in which the lobe is positioned within the body of the LAA and the disc abuts tissue at the opening of the LAA, the depth of the disc and the radius of curvature of the side surface of the disc are configured to prevent the disc from receding into the body of the LAA while preventing blood flow from stagnating along the proximal surface of the disc.

7. The system of claim 6, wherein the occluder is formed of nitinol.

8. The system of claim 7, wherein the disc is formed as a heat-set fabric.

9. The system of claim 7, wherein the disc includes a frame.

10. The system of claim 9, wherein the disc includes a fabric coupled to the frame.

11. A method for occluding a left atrial appendage (“LAA”), the method comprising:
providing an occluder comprising a lobe positioned at a distal end of the occluder, a disc positioned at a proximal end of the occluder, and a connecting segment that connects the disc to the lobe and spaces apart the disc from the lobe;
advancing the occluder to the LAA using a delivery system including a catheter and a delivery cable, wherein the occluder is in a collapsed condition within the catheter during the advancing;
deploying the occluder from the catheter so that the lobe self-expands into a body of the LAA and so that the disc self-expands to abut tissue at an opening of the LAA to occlude blood flow; and
de-coupling the occluder from the delivery cable,
wherein the disc includes a proximal surface including a proximal peripheral portion, and a distal surface including a distal peripheral portion, the proximal and distal peripheral portions extending in a proximal longitudinal direction after the disc self-expands, the distal peripheral portion of the distal surface separated from the proximal peripheral portion of the proximal surface by a depth such that an enclosed annular cavity is defined along a periphery of the disc between the proximal peripheral portion of the proximal surface and the distal peripheral portion of the distal surface, the depth being between 3.175 mm and 5.08 mm, wherein the disc includes a side surface extending between the proximal peripheral portion of the proximal surface and the distal peripheral portion of the distal surface, wherein the side surface includes a radius of curvature between 2 mm and 10 mm,
wherein, after deploying the occluder from the catheter, the depth of the disc and the radius of curvature of the side surface of the disc act to prevent the disc from receding into the body of the LAA while preventing blood flow from stagnating along the proximal surface of the disc.

12. The method of claim 11, wherein after deploying the occluder from the catheter, a radial center portion of the distal surface of the disc is positioned within the body of the LAA and the distal peripheral portion of the distal surface of the disc abuts tissue at the opening of the LAA.

13. The method of claim 11, wherein after deploying the occluder from the catheter, tension from the connecting segment tends to pull the distal surface of the disc away from the proximal surface of the disc.

14. The method of claim 11, wherein the occluder is formed of nitinol, the disc includes a frame and a fabric coupled to the frame.
